(19) United States
(12) Patent Application Publication
(10) Pub. No.: US 2021/0205812 A1
(43) Pub. Date: Jul. 8, 2021

(54) PARTICLE SEPARATION FROM WHOLE BLOOD

(71) Applicant: Hewlett-Packard Development Company, L.P., Spring, TX (US)

(72) Inventors: Viktor Shkolnikov, Palo Alto, CA (US); Caitlin DeJong, Palo Alto, CA (US); Daixi Xin, Palo Alto, CA (US)

(73) Assignee: Hewlett-Packard Development Company, L.P., Spring, TX (US)

(21) Appl. No.: 17/255,789

(22) PCT Filed: Sep. 24, 2018
(86) PCT No.: PCT/US2018/052465

§ 371 Date: Dec. 23, 2020

(51) Int. Cl. B01L 3/00; B03C 5/00; B03C 5/02; G01N 27/447

(52) U.S. Cl. CPC B01L 3/502761; B03C 5/005; B03C 5/026; B03C 2201/26; B01L 2400/0424; B01L 2200/0652; B01L 2200/027; G01N 27/44791

(57) ABSTRACT

Techniques for separating particles of interest from whole blood are disclosed. An example particle separation chip includes a first inlet on the particle separation chip for receiving whole blood and a second inlet on the particle separation chip for receiving a lysis buffer. The particle separation chip also includes a mixer to mix the whole blood with the lysis buffer to provide lysis of red blood cells in the whole blood. The particle separation chip also includes a buffer exchanger to exchange the lysis buffer for a dielectrophoresis buffer to produce a solution that enables dielectrophoretic separation of particles of interest. The particle separation chip also includes a separator coupled to an output of the buffer exchanger to separate the particles of interest from other particles in the solution via dielectrophoretic separation and deliver the particles of interest to an outlet on the particle separation chip.

### BACKGROUND

The separation of particles from blood enables a wide range of diagnostic capabilities. For example, particle separation can be used to separate rare cells such as cancer cells from blood to enable analysis of the cancer cells. Other particles that may be separated from blood include proteins, white blood cells, and others.

### DESCRIPTION OF THE DRAWINGS

Certain examples are described in the following detailed description and in reference to the following drawings.

FIG. 1 is a block diagram of a particle separation device, in accordance with examples.

FIG. 2A is a top view of a particle separation chip, in accordance with examples.

FIG. 2B is a side view of the particle separation chip, in accordance with examples.

FIG. 3 is a block diagram of a particle separation system, in accordance with examples.

FIG. 4 is a graph showing the relationship between the crossover frequency of cells and buffer conductivity, in accordance with examples.

FIG. 5 is a block diagram summarizing a method for separating particles of interest from whole blood, in accordance with examples.

### DETAILED DESCRIPTION

This disclosure is related to a new way of separating cells of interest from whole blood. More specifically, the present disclosure describes an integrated system for lysing red blood cells and performing dielectrophoretic (DEP) separation in one continuous flow. Dielectrophoresis is phenomenon in which a force is exerted on a dielectric particle when it is subjected to a non-uniform electric field. The force on the particle will depend on the size and polarization characteristics of the particle. These forces can be used for separating particles from bodily fluids, for example, cancer cells from blood.

Cells are not separated directly from untreated body fluids, because body fluids such as whole blood have an electrical conductivity that is too high for effective DEP separation. Thus, in an example, to isolate cells from whole blood via DEP, whole blood is first centrifuged and washed or sufficiently diluted. These steps are not automated and are liable to contaminate the sample and/or loose the rare cells from the sample.

The present disclosure describes a microfluidic chip that takes whole blood as an input, combines the blood with a red blood cell (RBC) lysis buffer to lyse the RBCs, then exchanges the buffer in the blood/lysate solution to a buffer with a particular conductivity and osmolality to enable DEP separation and not lyse cells of interest. The microfluidic chip then dilutes the cell flow to an appropriate cell volume fraction to enable DEP separation, and passes the cells to a section where dielectrophoretic force is applied orthogonal to the direction of the focused stream. The dielectrophoretic force moves the cells of interest into a particular channel, while moving the cells not of interest into another channel.

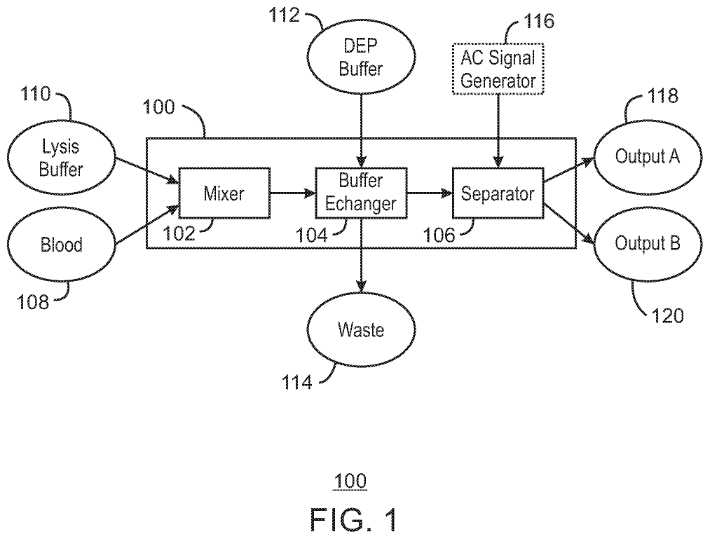

FIG. 1 is a block diagram of a particle separation device, in accordance with examples. The particle separation device 100 includes a mixer 102, a buffer exchanger 104, and a separator 106. The mixer 102, the buffer exchanger 104, and separator 106 may be included in single unified component, such as a single microfluid chip. However, in some embodiments, the mixer 102, buffer exchanger 104, and separator 106 can also be two or more separate components that are configured to be coupled together to enable fluid communication between the components.

The particle separation device 100 includes various inlets and outlets for receiving input fluids and delivering output fluids. For example, the particle separation device 100 includes an inlet for receiving blood 108. The blood may be whole blood, which is blood from which none of the components, such as plasma, platelets, or other blood cells have been removed. In some examples, the blood may be whole blood which has been drawn directly from the body and has not been treated in any manner.

The particle separation device 100 also includes an inlet for receiving a lysis buffer 110. The lysis buffer may be any one of a number of buffers capable of lysing red blood cells. Particular types of lysis buffer are described further below. The lysis buffer 110 and the red blood cells 108 are introduced into a mixer 102. The mixer 102 may be a passive mixer, such as a serpentine mixer. The mixer 102 enables the lysis buffer to lyse the red blood cells without lysing other cell types that may be of interest. The lysis of blood cells is a time dependent process in which the red blood cells tend to be more sensitive to the lyse buffer compared to other cells, such as white blood cells, cancer cells, and the others. Accordingly, the mixer 102 may be sized such that under a specific flow rate, the lysis buffer and the blood will mix for a period of time sufficient for lysis of the red blood cells without lysing the other cells.

The output of the mixer 102 is coupled to the input of the buffer exchanger 104. The flow of whole blood passes from the mixer 102 to the buffer exchanger 104 after the lysing buffer has had time to lyse most or all of the red blood cells in the whole blood. The buffer exchanger removes the lysing buffer and replaces it with a dielectrophoresis (DEP) buffer 112. The DEP buffer is isosmotic relative to blood to avoid lysing cells of interest. Replacing the lysing buffer with the DEP buffer terminates the lysing process to ensure that the cells of interest are not lysed. The introduction of the DEP buffer produces a solution that has a level of osmolality and conductivity suitable for dielectrophoretic separation.

In some examples, the buffer exchanger operates according to a dialysis process, in which the DEP buffer 112 is used as a dialysate. The buffer exchanger outputs waste 114, which is the used dialysate from the dialysis process. The waste carries away at least some of the byproducts of the red blood cell lysing process. In some examples, an additional supply of the DEP buffer may be introduced into the buffer exchanged solution to dilute the cell flow to an appropriate cell volume fraction to enable DEP separation. The output of the buffer exchanger is a solution of blood and DEP buffer.

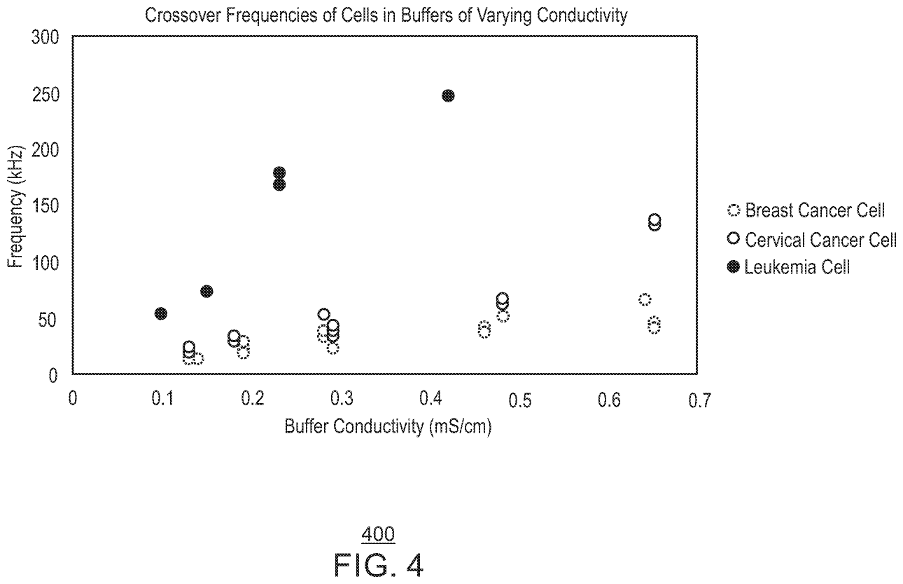

The resulting solution is output from the buffer exchanger to the separator 106, which may be any type of dielectrophoretic separator. The separator 106 includes electrodes, which are coupled to an AC signal generator 116 to generate an electromagnetic field within the separator 106. The electric field generates a dielectrophoretic force on the cells and other particles within the solution. Proper selection of the conductivity of the DEP buffer and the frequency of the AC signal, will cause different particle types to experience a different dielectrophoretic force. As a result, particles of interest will be moved to one channel and other particles will be moved to another channel. The selection of the DEP buffer conductivity and AC signal frequency is described further below in relation to FIG. 4.

The separator 106 shown in FIG. 1 includes two outlets for outputting the separated particles, referred to as output A 118 and output B 120. As an example, output A 118 may receive particles of interest and output B 120 may receive all of the other particles, which may not be of interest and can be considered waste. However, various other configurations are also possible. For example, in some cases, there may be two particles of interest, one that is transferred to output A 118 and one that is transferred to output B 120. Additionally, although two outputs are shown in FIG. 1, the separator 106 may also include additional outputs for receiving additional particles of interest and/or waste products.

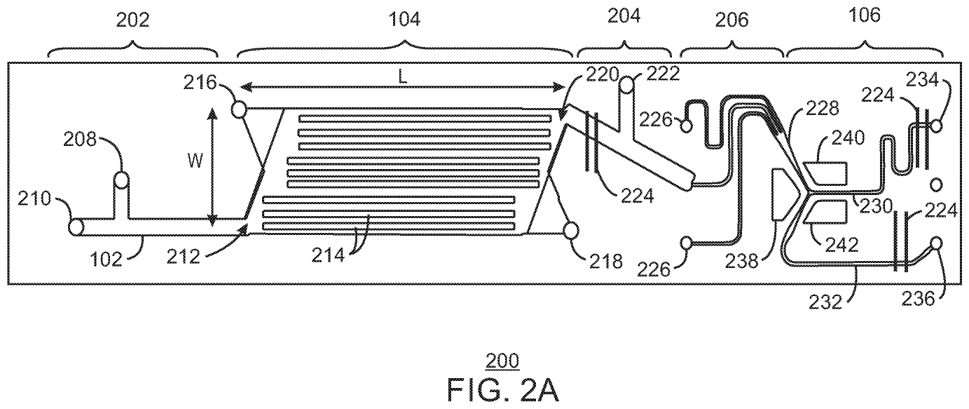

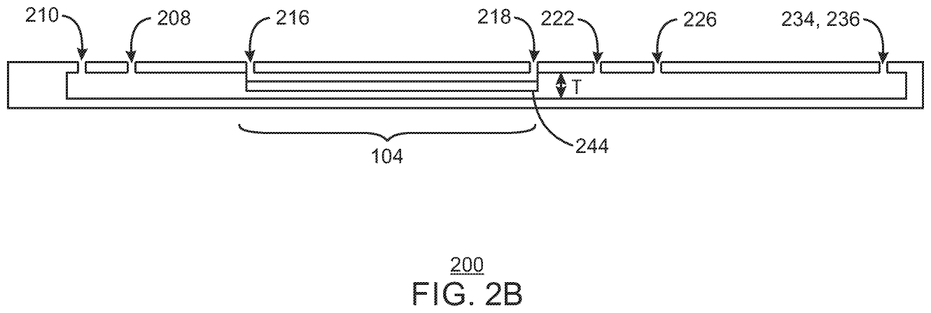

It is to be understood that the block diagram of FIG. 1 is not intended to indicate that the particle separation device 100 is to include all of the components shown in FIG. 1. Rather, the particle separation device can include fewer or additional components not illustrated in FIG. 1. A more detailed example of a particle separation device is described below with reference to FIGS. 2A and 2B.

FIG. 2A is a top view of a particle separation chip, in accordance with examples. The particle separation chip 200 is an example of the particle separation device 100 shown in FIG. 1. The particle separation chip 200 includes a number of sections, including the RBC lysis section 202, the buffer exchanger 104, a dilution section 204, a particle focuser 206, and the separator 106.

The RBC lysis section 202 includes a first inlet 208 for receiving a lysis buffer and a second inlet 201 for receiving whole blood. The whole blood and the lysis buffer combine within the mixer 102. As mentioned above, the mixer 102 may be serpentine mixer or other type of mixer that enables the lysis buffer and the red blood cells to mix for a period of time sufficient to lyse the red blood cells without lysing other cells or particles, including the cells or particles of interest. Examples of commercially available lysis buffers are described in Table 1 below. The lysis buffer injected into the inlet 208, may be one of the lysis buffers described in table 1, or other lysis buffer.

TABLE 1
Lysis Buffers

| Company or Recipe | Catalog # | Reagent | Concentration |
|---|---|---|---|
| eBioscience RBC Lysis | 00-4300- | Ammonium | 7-13% |
| Buffer (Multi-species) 10X | 54 | chloride | |
| eBioscience RBC Lysis | 00-4333- | Ammonium | 0.7-1.3% |
| Buffer (Multi-species) 1X | 57 | chloride | |
| ACK Lysing Buffer (1X) | A1049201 | Ammonium | 154.95328 |
| | | Chloride | |
| | | Potassium | 9.99001 |
| | | Bicarbonate | |
| | | EDTA | 0.09946237 |
| BioLegend RBC Lysis | 420301 | Ammonium | Not |
| Buffer (10X) | | chloride, | published |
| | | potassium | |
| | | carbonate, | |
| | | EDTA | |
| STEMCELL EasyStep Red | 20110 or | Ammonium | 8.25% |
| Blood Cell | 20120 | Chloride | |
| Lysis Buffer (10X) | | | |
| CSH RBC lysis buffer | N/A | NH4Cl | 155 mM |
| | | NaHCO3 | 12 mM |
| | | EDTA | 0.1 mM |

After leaving the mixer, the whole blood enters the buffer exchanger 104 at the buffer exchanger input 212. The buffer exchanger 104 includes a membrane through which cells cannot pass but smaller components, such as ions, sugars, and proteins can freely diffuse. The buffer exchanger 104 is discussed in greater detail in relation to FIG. 2B, which shows a side view of the buffer exchanger 104. As shown in FIG. 2A, the buffer exchanger includes a membrane support grid 214. The DEP buffer may be injected at the DEP buffer input port 216 and flow across the membrane to exit at the waste port 218. It will be appreciated that the buffer exchanger can be operated in a co-current or counter current exchange mode. In the counter current exchange mode, the DEP buffer is injected at port 218 and flows across the membrane to exit at the port 216. An example DEP buffer chemistry is described further in relation to FIG. 4.

The buffer exchanged blood at the output 220 of the buffer exchanger 104 may be referred to herein as a cell containing solution. The amount of lysis buffer in the cell containing solution will be substantially reduced or eliminated, thus preventing further lysis which could otherwise effect the cells of interest. The cell containing solution at the output of the buffer exchanger 104 will also have a substantial amount of the red blood cells lysed and eliminated. In some examples, the red blood cell concentration may be reduced from about 10⁹ red blood cells per milliliter to about 10⁶ red blood cells per milliliter. Thus, to analyze one milliliter of blood, the device needs to sort on only 10⁶ cells, rather than 10⁹ cells, increasing the throughput of the device 1000 fold.

After the buffer exchanger 104, the cell containing solution may enter the dilution section 204. At the dilution section 204, additional DEP buffer is injected through port 222 into the cell containing solution to further dilute the cell containing solution. In some examples, the dilution may achieve a cell concentration of less than one percent (cell volume/buffer volume). In some examples, a cell counter 224 may be disposed between the output 220 of the buffer exchanger and the port 22 of the dilution section 204. The cell counter 224 may be used to count the cells exiting the buffer exchanger 204 to determine the cell concentration. To achieve a target cell concentration, the cell concentration of the solution exiting the buffer exchanger 220 may be measured using the cell counter 224, and the measured cell concentration may be used to control the amount of the DEP buffer injected into the port 22 of the dilution section 204.

The diluted cell containing solution exits the dilution section 204 and enters the particle focuser 206. The particle focuser includes two DEP buffer inlets 226. The DEP buffer injected into the DEP buffer inlets meet with the cell containing solution at the inlet passage 228 of the separator 106. The particle focuser 206 focuses the particles entrained in the cell containing solution into a laminar flow within the inlet passage 228 prior to separation. In the example shown in FIG. 2, the particle focuser 206 is a hydrodynamic focuser that uses first and second sheath flows of the DEP buffer solution to sandwich the cell containing solution to provide the laminar flow of particles through the inlet passage 228. In other implementations, other particle focusers, such as free flow negative dielectrophoresis particle focusers and free flow isotachophoresis particle focusers, may be used. Focusing the particles improves the accuracy of the DEP separation by improving the consistency of the DEP forces exerted on the particles. In some examples, the focuser 206 may be eliminated and the cell containing solution can enter directly from the dilution section to the DEP separator 106.

The focused particle stream enters the inlet passage 228 of the DEP separator 106. In the example shown in FIG. 2, the separator 106 includes the inlet passage 228, a first separation passage 230, a second separation passage 232. The separation passages 230 and 232 comprise channels, such as microfluidic channels that extend from and branch off of inlet passage 228. The separation passages 230 and 232 lead to output wells 234 and 236 where the separated particles or cells may be collected and analyzed. Although passages 234 and 236 are illustrated as branching off of inlet passage 228 at angles of approximately 135 degrees, it should be appreciated that passages 234 and 236 may extend at other angles from inlet passage 228. Additionally, the separator 106 may include additional separation passages and additional output wells compared to what is shown on FIG. 2. For example, in some implementations, particles directed to separation passage 230 and separation passage 232 may be further separated downstream.

The example separator 106 also includes electrodes 238, 240, and 242, which create electric fields across the passages 228, 230, and 232. The electrodes 238, 240, and 242 extend in a single plane such that they produce electric fields that extend in the same plane as that of passages 228, 230, and 232. In the example shown in FIG. 2, electrode 238 is a ground electrode that extends along alongside passages 228 and 232, the electrode 240 extends alongside passages 228 and 230, and the electrode 242 extends alongside passages 230 and 232. The electrodes 240 the electrode 242 may be of opposite polarity. For example, electrode 240 may be a positive electrode and electrode 242 may be a negative electrode, or vise versa. Each of the electrodes 238, 240, and 242 may be a continuous electrode or may be formed by multiple separate elements connected to ground or a source of electrical current, such as an alternating frequency electric current source.

In some examples, electrodes 238 and 240 are separated by a distance across inlet passage 228 by distance of at least 10 times a diameter of a target particle to be separated. Likewise, electrodes 240 and 242 as well as electrodes 238 and 242 are also separated by distance across separation passages 230 and 232, respectively, by a distance of at least 10 times a diameter of the target particles being separated.

The electrodes 238, 240, and 242 apply alternating current (AC) electric fields in a plane to the stream of fluid entrained particles. The frequency of the AC fields may be selected depending on the particles being targeted for separation, as explained further in relation to FIG. 4. The electric fields exert dielectrophoretic forces in a plane on the particles, the same plane in which inlet passage 228 and separation passages 230 and 232 extend and the same plane in which the electric fields extend. The particles are separated based upon their different responses to the dielectrophoretic forces as a result of their different size and electric polarizability. The dielectrophoretic forces divert a first subset of the particles in the stream into the first separation passage 230 and divert a second subset of the particles in the stream into the second separation passage 232. In some examples, each separation passage 230 and 232 may be associated with a cell counter 224 that counts the number of cells entering each respective output well 234 and 236.

FIG. 2B is a side view of the particle separation chip, in accordance with examples. FIG. 2B the membrane of the buffer exchanger 104 is shown with the reference number 244. The membrane 244 may be a cellulose dialysis membrane with a 1000 kilo Dalton (kDa) cut off. On one of side of a membrane 244 is a channel for whole blood flow, while on the other side is a channel for DEP dialysis buffer flow. This buffer is isosmotic relative to blood as not to lyse cells of interest such as nucleated circulating tumor cells, nucleated red blood cells, and white blood cells. The buffer exchanger can be operated in a co-current or counter current exchange modes.

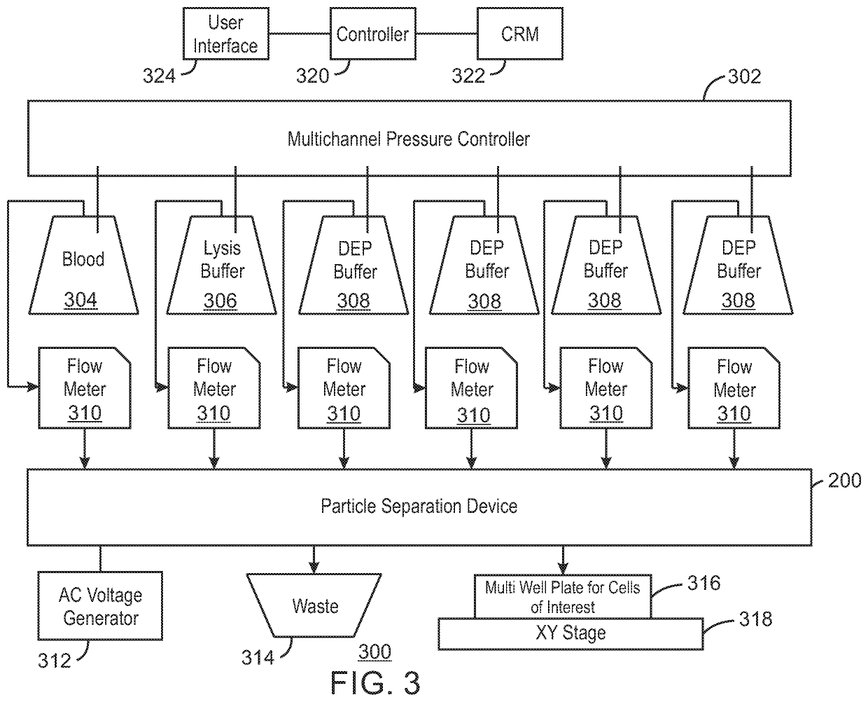

The particle separation chip 200 may be coupled to a particle separation system such as the particle separation system shown in FIG. 3. The particle separation system controls the flow of fluids, such as the whole blood and the various buffers, through the particle separation chip at specified flow rates. The flow rates will depend on various factors including the dimensions of the various components of the particle separation chip 200.

For example, the buffer exchanger 104 may be dimensioned and controlled to provide a cell containing solution at the output of the buffer exchanger with a conductivity of approximately 0.3 milliSiemens per centimeter (mS/cm). The conductivity of whole blood is approximately 15-20 mS/cm, and the conductivity of the blood-lysis solution is substantially similar. For example, 100 mM ammonium chloride has a conductivity of 13 mS/cm. Thus, to achieve 0.3 mS/cm for the cell containing solution, the buffer exchanger may dialyze the whole blood with a volume of DEP buffer equal to approximately 100 times the volume of the whole blood. Accordingly, the flow rate of the DEP buffer entering the buffer exchanger 104 at the input 216 (referred to herein as Q_{D}) will be greater than or equal to 100 times the flow rate of the whole blood entering the buffer exchanger at the input 212 (referred to herein as Q_{BL}).

The residence time, t_{resBL}, of the blood in the buffer exchanger 104 may be computed according to the following formula:

$${t}_{resBL} = \frac{( \frac{T}{2} ) * W * L}{{Q}_{BL}}$$

In the above formula, W is the width of the buffer exchanger, L is the length of the buffer exchanger, and T/2 is the overall thickness of the whole blood channel in the buffer exchanger as shown in FIGS. 2A and 2B. To achieve the desired blood conductivity, the residence time, t_{resBL}, should be equal or greater than the total time for ions and sugars to diffuse from the blood into the buffer and from the buffer into the blood, t_{diff}, which may be determined according to the following formula:

t_{diff}=t_{diffL}+t_{diffM}

In the above formula, t_{diffL} represents the total time to diffuse across the liquid, and t_{diffM} represents the total time to diffuse across the membrane. Additionally, t_{diffL} may be determined according to the following formula:

$${t}_{diffL} = \frac{{( \frac{T}{2} )}^{2}}{D}$$

In the above formula, D is the diffusivity of the slowest diffusing species. For sucrose, D=5×10⁻¹⁰ m²/s. The time to diffuse through a membrane may be modeled as a first approximation as t_{diffM}=k/D where k is a constant that scales as the membrane thickness and permeability of the membrane. Depending on the membrane, either t_{diffM} or t_{diffL} dominates. In some examples, the membrane thickness may be selected so that t_{diffM} and t_{diffL} are comparable, so that t_{diff}=2t_{diffL}. As stated above, for the buffer exchanger to work properly, t_{resBL}≥t_{diff}. This lead to the following relationship:

$$\frac{( \frac{T}{2} ) * W * L}{{Q}_{BL}} = 2 \frac{{( \frac{T}{2} )}^{2}}{D}$$

Simplification of the above formula yields:

$$T = \frac{W * L}{{Q}_{BL}} D$$

Based on the above formula, an example buffer exchanger 104 may be constructed and operated according to the values shown in Table 2 below.

TABLE 2
Example Buffer Exchanger Design

| Parameter | Value |
|---|---|
| Q_{BL} | 0.005 ml/min |
| Q_{D} | 0.5 ml/min |
| W | 1 cm |
| L | 2 cm |
| T | 1.2 mm (or smaller) |
| k | 0.36 mm² |

The particle separation chip 200 may be manufactured according to any suitable manufacturing technique. In some examples, the particle separation chip 200 may be fabricated as a silicon or polymer substrate with glass plate coupled to the top surface. Suitable polymers may include cyclic olefin copolymer (COC), polycarbonate, acrylic, Teflon, nitrocellulose, poly ether ketone (PEEK), and others. Channels in the substrate may formed by cutting, ablation, etching, or other material removal processes carried out on the layer or layers of the material forming substrate. The channels may also be formed by selective deposition, such as printing or additive manufacturing processes carried out upon an underlying base layer or platform. Channels in the substrate may also be hot embossed or formed through injection molding to form a molded interconnect device (MID). The electrodes 238, 240, and 242 may be formed by vapor deposition or sputtering of a conductive material such as copper or gold, as well as other suitable techniques.

FIG. 3 is a block diagram of a particle separation system, in accordance with examples. The particle separation system 300 includes the particle separation device 200 as well as a variety of hardware that can be controlled to deliver fluids to the particle separation device and direct the processes performed to achieve the particle separation. The particle separation device 200 may be in the form of a cartridge or chip that may be inserted into a receptacle of the particle separation system 300.

The example particle separation system 300 shown in FIG. 3 includes a multichannel pressure controller 302 to control delivery of the various fluids to the particle separation device 200. The multichannel pressure controller 302 is coupled to a number of vessels that contain the fluids, including a blood vessel 304 that contains the whole blood, a lysis buffer vessel 306, and a number of DEP buffer vessels 308. The multichannel pressure controller 302 controls the injection of fluids from the vessels 304, 306, and 308 into the particle separation device 200. For example, the multichannel pressure controller 302 may operate by delivering a pressurized gas, such as air or Nitrogen, into a head space of the vessels 304, 306, and 308. Each output of the multichannel pressure controller 302 may be controlled separately to deliver different rates of fluid injection depending on the design details of a particular implementation.

Additionally, each vessel 304, 306, and 308 may be coupled to a flow meter 310 that senses the actual flow rate. The flow meters 310 may be of any suitable type, including thermal pulse flow meters and others. The flow meters 310 may provide a feedback signal corresponding to the measured flow rate back to the multichannel pressure controller 302. This feedback loops enables the multichannel pressure controller 302 to accurately control the flow rates.

The particle separation system 300 also includes an AC voltage generator 312 coupled to the electrodes 238, 240, and 242 (FIG. 2A) of the particle separation device 200. The AC voltage generator 312 generates the AC signal that generates the dielectrophoretic forces within the particle separation device 200. The particle separation device 200 may also be coupled to a waste container 314 that receives the waste buffer from the buffer exchanger 104 (FIG. 2A). In some examples, the particle separation system 300 also includes a well plate 316 for collecting cells of interest. The well plate 316 may be include multiple wells for collecting cells of interest and may be coupled to a mobile platform 318 for directing the cells of interest to selected wells.

The particle separation system 300 may also include a system controller 320 which directs the actions of the multichannel pressure controller 302, the AC voltage generator 312, and the mobile platform 318. The controller may also receive feedback from the cell counters 224 (FIG. 2A) to facilitate operation of the particle separation system 300, for example, to achieve the proper conductivity for the cell containing solution at the output of the buffer exchanger 104 (FIG. 2A) to direct the cells of interest to selected wells of the well plate 316 and the like.

The controller 302 may include a processor which may be a microprocessor, a multi-core processor, a multithreaded processor, an ultra-low voltage processor, an embedded processor, or other type of processor. The processor 1202 may be an integrated microcontroller in which the processor 1202 and other components are formed on a single integrated circuit board, or a single integrated circuit, such a system on a chip (SoC). As an example, the processor 1202 may include a processor from the Intel® Corporation of Santa Clara, Calif., such as a Quark™, an Atom™, an i3, an i5, an i7, or an MCU-class processor. Other processors that may be used may be obtained from Advanced Micro Devices, Inc. (AMD) of Sunnyvale, Calif., a MIPS-based design from MIPS Technologies, Inc. of Sunnyvale, Calif., an ARM-based design licensed from ARM Holdings, Ltd. or customer thereof, or their licensees or adopters. The processors may include units such as an A5-A10 processor from Apple® Inc., a Snapdragon™ processor from Qualcomm® Technologies, Inc., or an OMAP™ processor from Texas Instruments, Inc.

The controller 302 may communicate with a computer readable medium 322, which may include any type and number of memory devices provide for a given amount of system memory. The computer readable medium 322 may be implemented using volatile or non-volatile memory devices such as Random Access Memory (RAM), a solid-state drive (SSD), flash memory, such as SD cards, microSD cards, xD picture cards, USB flash drives, a hard disk drive, and the like.

The controller 320 can also include or be coupled to a user interface 324. For example, the user interface 324 may include a display panel and an input device, such as a touch screen or keypad, among others. The user interface 324 enables a user of the particle separation system 300 to interact with and implement the functionality of the particle separation system 300 as described herein.

FIG. 4 is a graph showing the relationship between the crossover frequency of cells and buffer conductivity, in accordance with examples. As explained above, the dielectrophoretic separation is a process wherein an electric field generates a dielectrophoretic force on cells and other particles within the solution. The degree and direction of the dielectrophoretic force depends on the conductivity of the cell containing solution, the frequency of the AC signal, and the electrical properties of the cells. Thus, proper selection of the conductivity of the cell containing solution and frequency of the AC signal enables an operator of the particle separation system 300 to target particular types of cells.

The graph 400 shows the crossover frequencies of various cells types in buffers of varying conductivity. In the graph 400, the X-axis represents the buffer conductivity in milliseimens per centimeter, and the Y-axis represents frequency in kilohertz. The crossover frequency is plotted for various cell types and various buffer conductivities. The crossover frequency is the frequency at which the direction of dielectrophoretic force reverses to the opposite direction. For example, a leukemia cell in a DEP buffer with a conductivity of 0.1 milliseimens per centimeter exhibits a crossover frequency of about 55 kHz. Above that frequency, the dielectrophoretic force will be in one direction and below that frequency, the dielectrophoretic force will be in the opposite direction. This information can be used to control the direction in which targeted cells are directed based on the AC frequency and the DEP buffer conductivity. As a result, particles of interest can be moved to one channel and other particles can be moved to another channel.

To ensure cell viability and general health, the DEP buffer may be a pH7 phosphate-based buffer with a variety of components to decrease cell stress. For example, sugars such as sucrose and dextrose may be added to balance the osmolarity of the cell containing solution and provide an energy source for the cells. Other components that may be added include pluronic acid, which protects cells from flow damage, and Bis(trimethylsilyl)acetamide (BSA) to minimize cell sticking. Additionally, the DEP buffer may include a catalase to reduce free radical production and subsequent damage. The DEP buffer may also include calcium acetate and magnesium acetate to stabilize membrane integrity. One example of a DEP buffer that may be used in the described techniques includes 9.5% sucrose, 0.1 mg/ml dextrose, 0.1% pluronic F68, 0.1% bovine serum albumin, 1 mM phosphate buffer pH 7, 0.1 mM CaAcetate, 0.5 mM MgAcetate, and 100 units/ml catalase. The conductivity of the DEP buffer may be varied by varying the concentration of the phosphate buffer, where a higher concentration of phosphate buffer results in higher conductivity and vice versa.

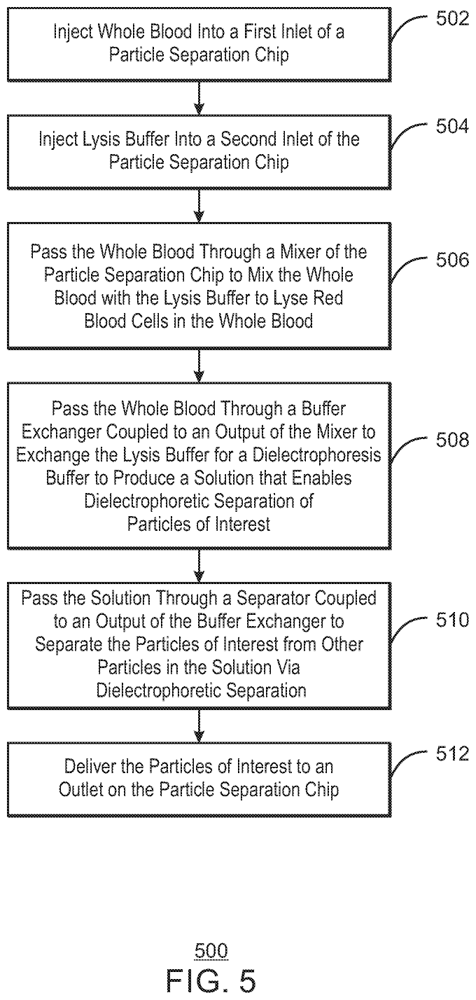

FIG. 5 is a block diagram summarizing a method for separating particles of interest from whole blood, in accordance with examples. The method 500 may be performed by a particle separation system such as the particle separation system 300 described above in relation to FIG. 3. The method 500 may begin at block 502.

At block 502, whole blood is injected into a first inlet of a particle separation chip. At block 504, a lysis buffer is injected into a second inlet of the particle separation chip. At block 506, the whole blood is passed through a mixer of the particle separation chip. The mixer mixes the whole blood with the lysis buffer to lyse red blood cells in the whole blood.

At block 508, the whole blood is passed through a buffer exchanger coupled to an output of the mixer to exchange the lysis buffer for a dielectrophoresis buffer to produce a solution that enables dielectrophoretic separation of particles of interest. In some examples, the buffer exchanger includes two channels separated by a semipermeable dialysis membrane. The whole blood flows through one channel and the dielectrophoresis buffer flows through the other channel.

At block 510, the solution is passed through a separator coupled to an output of the buffer exchanger to separate the particles of interest from other particles in the solution via dielectrophoretic separation.

At block 512, the particles of interest are delivered to an outlet on the particle separation chip. In some examples, the separator includes a particle focuser that receives an additional supply of the dielectrophoresis buffer and focuses the particles of interest into a laminar flow. Additionally, the separator may operate by applying an AC electric field to the cell containing solution in the separator. The frequency of the AC electric field may be selected to target the particles of interest.

The method 500 should not be interpreted as meaning that the blocks are necessarily performed in the order shown. Furthermore, fewer or greater actions can be included in the method 500 depending on the design considerations of a particular implementation. For example, another supply of the dielectrophoresis buffer may be injected at the output of the buffer exchanger to further dilute the solution.

While the present techniques may be susceptible to various modifications and alternative forms, the examples discussed above have been shown by way of example. It is to be understood that the techniques are not intended to be limited to the particular examples disclosed herein. Indeed, the present techniques include all alternatives, modifications, and equivalents falling within the scope of the present techniques.

What is claimed is:

1. A particle separation chip, comprising:
a first inlet on the particle separation chip for receiving whole blood;
a second inlet on the particle separation chip for receiving a lysis buffer;
a mixer to mix the whole blood with the lysis buffer to provide lysis of red blood cells in the whole blood;
a buffer exchanger coupled to an output of the mixer to exchange the lysis buffer for a dielectrophoresis buffer to produce a solution that enables dielectrophoretic separation of particles of interest; and
a separator coupled to an output of the buffer exchanger to separate the particles of interest from other particles in the solution via dielectrophoretic separation and deliver the particles of interest to an outlet on the particle separation chip.

2. The particle separation chip of claim 1, wherein the buffer exchanger comprises:
a first channel to pass a flow of the whole blood;
a second channel to pass a flow of the dielectrophoresis buffer; and
a semipermeable membrane separating the first channel and the second channel.

3. The particle separation chip of claim 1, comprising a third inlet on the particle separation chip to receive a second supply of the dielectrophoresis buffer at the output of the buffer exchanger to further dilute the solution.

4. The particle separation chip of claim 1, comprising a fourth inlet on the particle separation chip to receive a third supply of the dielectrophoresis buffer, wherein the third supply of the dielectrophoresis buffer is delivered to a particle focuser to focus the particles of interest into a laminar flow.

5. The particle separation chip of claim 1, wherein the separator comprises electrodes to apply an alternating current (AC) electric field to the solution, wherein a frequency of the AC electric field is adjustable to target the particles of interest.

6. A method of separating particles of interest from whole blood, comprising:
injecting whole blood into a first inlet of a particle separation chip;
injecting a lysis buffer into a second inlet of the particle separation chip;
passing the whole blood through a mixer of the particle separation chip to mix the whole blood with the lysis buffer to lyse red blood cells in the whole blood;
passing the whole blood through a buffer exchanger coupled to an output of the mixer to exchange the lysis buffer for a dielectrophoresis buffer to produce a solution that enables dielectrophoretic separation of particles of interest; and
passing the solution through a separator coupled to an output of the buffer exchanger to separate the particles of interest from other particles in the solution via dielectrophoretic separation; and
delivering the particles of interest to an outlet on the particle separation chip.

7. The method of claim 6, wherein passing the whole blood through a buffer exchanger comprises:
passing the whole blood through a first channel; and
passing the dielectrophoresis buffer through a second channel separated from the first channel by a semipermeable membrane.

8. The method of claim 6, comprising injecting a second supply of the dielectrophoresis buffer at the output of the buffer exchanger to further dilute the solution.

9. The method of claim 6, comprising injecting a third supply of the dielectrophoresis buffer into a particle focuser to focus the particles of interest into a laminar flow.

10. The method of claim 6, comprising applying an alternating current (AC) electric field to the solution in the separator, wherein a frequency of the AC electric field is selected to target the particles of interest.

11. A particle separation system, comprising:
a receptacle to receive a particle separation chip;
a signal generator to provide an alternating current (AC) electrical signal to electrodes of the particle separation chip to generate a dielectrophoretic force;
a fluid delivery system configured to provide a plurality of fluids to the particle separation chip, wherein the fluid delivery system is to:
inject whole blood into a first inlet of the particle separation chip;
inject a lysis buffer into a second inlet of the particle separation chip, wherein the lysis buffer is to lyse red blood cells in the whole blood; and
inject a dielectrophoresis buffer into a third inlet of the particle separation chip to dialyze the whole blood to replace the lysis buffer with the dielectrophoresis buffer to produce a solution that enables dielectrophoretic separation of particles of interest from the whole blood as a result of the dielectrophoretic force.

12. The particle separation system of claim 11, wherein the third inlet is coupled to a buffer exchanger comprising:
a first channel to pass a flow of the whole blood;
a second channel to pass a flow of the dielectrophoresis buffer; and
a semipermeable membrane separating the first channel and the second channel.

13. The particle separation system of claim 11, comprising a fourth inlet on the particle separation chip to receive a second supply of the dielectrophoresis buffer to further dilute the solution after dialyzing the whole blood.

14. The particle separation system of claim 11, comprising a fifth inlet on the particle separation chip to receive a third supply of the dielectrophoresis buffer, wherein the third supply of the dielectrophoresis buffer is delivered to a particle focuser to focus the particles of interest into a laminar flow.

15. The particle separation system of claim 11, wherein the AC electric field is adjustable to target the particles of interest.
